(19) United States
(12) Patent Application Publication
(10) Pub. No.: US 2014/0187959 A1
(43) Pub. Date: Jul. 3, 2014

(54) Ultrasound Transducers and Methods of Manufacturing Same

(71) Applicant: Volcano Corporation, San Diego, CA (US)

(72) Inventor: Paul Douglas Corl, Palo Alto, CA (US)

(73) Assignee: Volcano Corporation, San Diego, CA (US)

(21) Appl. No.: 14/139,564

(22) Filed: Dec. 23, 2013

Related U.S. Application Data
(60) Provisional application No. 61/747,453, filed on Dec. 31, 2012.

(51) Int. Cl. A61B 8/00

(52) U.S. Cl. CPC A61B 8/4483
USPC 600/462; 600/459; 29/594

(57) ABSTRACT

The present disclosure provides various embodiments of an ultrasound transducer for use in intravascular ultrasound (IVUS) imaging. An exemplary ultrasound transducer includes substrate having a first surface and a second surface opposite the first surface. A well is disposed in the substrate that extends from the first surface to the second surface, where a well surface of the substrate defines a shape of the well. The well surface has a greater surface area than a cylindrically-shaped well surface. The ultrasound transducer further includes an arcuate-shaped transducer membrane is positioned within the well adjacent to the first surface of the substrate, and a backing material is disposed in the well that physically contacts the well surface and the transducer membrane. The backing material secures the arcuate-shape of the transducer membrane.

### CROSS-REFERENCE TO RELATED APPLICATIONS

The present application claims priority to and the benefit of U.S. Provisional Patent Application No. 61/747,453, filed Dec. 31, 2012, which is hereby incorporated by reference in its entirety.

### TECHNICAL FIELD

The present disclosure relates generally to intravascular ultrasound (IVUS) imaging, and in particular, to an IVUS ultrasound transducer, such as a piezoelectric micromachined ultrasound transducer (PMUT), used for IVUS imaging.

### BACKGROUND

Intravascular ultrasound (IVUS) imaging is widely used in interventional cardiology as a diagnostic tool for assessing a vessel, such as an artery, within the human body to determine the need for treatment, to guide intervention, and/or to assess its effectiveness. An IVUS imaging system uses ultrasound echoes to form a cross-sectional image of the vessel of interest. Typically, IVUS imaging uses a transducer on an IVUS catheter that both emits ultrasound signals (waves) and receives the reflected ultrasound signals. The emitted ultrasound signals (often referred to as ultrasound pulses) pass easily through most tissues and blood, but they are partially reflected by discontinuities arising from tissue structures (such as the various layers of the vessel wall), red blood cells, and other features of interest. The IVUS imaging system, which is connected to the IVUS catheter by way of a patient interface module, processes the received ultrasound signals (often referred to as ultrasound echoes) to produce a cross-sectional image of the vessel where the IVUS catheter is located.

There are primarily two types of IVUS catheters in common use today: solid-state and rotational. An exemplary solid-state IVUS catheter uses an array of transducers (typically 64) distributed around a circumference of the catheter and connected to an electronic multiplexer circuit. The multiplexer circuit selects transducers from the array for transmitting ultrasound signals and receiving reflected ultrasound signals. By stepping through a sequence of transmit-receive transducer pairs, the solid-state IVUS catheter can synthesize the effect of a mechanically scanned transducer element, but without moving parts. Since there is no rotating mechanical element, the transducer array can be placed in direct contact with blood and vessel tissue with minimal risk of vessel trauma, and the solid-state scanner can be wired directly to the IVUS imaging system with a simple electrical cable and a standard detachable electrical connector.

An exemplary rotational IVUS catheter includes a single transducer located at a tip of a flexible driveshaft that spins inside a sheath inserted into the vessel of interest. The transducer is typically oriented such that the ultrasound signals propagate generally perpendicular to an axis of the IVUS catheter. In the typical rotational IVUS catheter, a fluid-filled (e.g., saline-filled) sheath protects the vessel tissue from the spinning transducer and driveshaft while permitting ultrasound signals to freely propagate from the transducer into the tissue and back. As the driveshaft rotates (for example, at 30 revolutions per second), the transducer is periodically excited with a high voltage pulse to emit a short burst of ultrasound. The ultrasound signals are emitted from the transducer, through the fluid-filled sheath and sheath wall, in a direction generally perpendicular to an axis of rotation of the driveshaft. The same transducer then listens for returning ultrasound signals reflected from various tissue structures, and the IVUS imaging system assembles a two dimensional image of the vessel cross-section from a sequence of several hundred of these ultrasound pulse/echo acquisition sequences occurring during a single revolution of the transducer.

An exemplary ultrasound transducer used in IVUS catheters is a piezoelectric micromachined ultrasound transducer (PMUT), which includes a polymer piezoelectric membrane, such as that disclosed in U.S. Pat. No. 6,641,540, hereby incorporated by reference in its entirety. The PMUT transducer can provide greater than 100% bandwidth for optimum resolution in a radial direction, and a spherically-focused aperture for optimum azimuthal and elevation resolution. The PMUT transducer typically includes a backing material disposed within a well and located on a backside of the polymer piezoelectric membrane. The backing material secures the membrane within the well and prevents (or reduces) noise caused by acoustic energy reflections from structures or interfaces of the PMUT transducer assembly. It has been observed that during fabrication and operation of the PMUT transducer, the backing material may become dislodged and/or removed from the well, modifying the mechanical strength and acoustic characteristics of the backing material. Such modification in the characteristics of the backing material degrades performance of the PMUT transducer. Accordingly, there remains a need for improved PMUT transducers for use in IVUS imaging and associated devices, systems, and methods of manufacturing.

### SUMMARY

The present disclosure provides various embodiments of an ultrasound transducer for use in intravascular ultrasound (IVUS) imaging. An exemplary ultrasound transducer includes a substrate having a first surface and a second surface opposite the first surface. A well is disposed in the substrate that extends from the first surface to the second surface, where a well surface of the substrate defines a shape of the well. The well surface has a greater surface area than a cylindrically-shaped well surface. The ultrasound transducer further includes an arcuate-shaped transducer membrane positioned within the well adjacent to the first surface of the substrate, and a backing material disposed in the well that physically contacts the well surface and the transducer membrane. The backing material secures the arcuate-shape of the transducer membrane. In some implementations, the ultrasound transducer is implemented in an IVUS imaging device. An exemplary IVUS imaging device includes an elongate flexible member having a proximal end portion and a distal end portion. The ultrasound transducer is coupled to the distal end portion of the flexible elongate member in some instances. In some embodiments, an integrated circuit is coupled to the distal end portion of the flexible elongate member and electrically coupled to the PMUT. An interface module may be coupled with the proximal end portion of the flexible elongate member, and an image processing component may be in communication with the interface module.

Both the foregoing general description and the following detailed description are exemplary and explanatory in nature and are intended to provide an understanding of the present disclosure without limiting the scope of the present disclosure. In that regard, additional aspects, features, and advantages of the present disclosure will become apparent to one skilled in the art from the following detailed description.

### BRIEF DESCRIPTIONS OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals and/or letters in the various examples. This repetition is for the purpose of simplicity and clarity and does not in itself dictate a relationship between the various embodiments and/or configurations discussed.

FIG. 1 is a schematic illustration of an intravascular ultrasound (IVUS) imaging system according to various aspects of the present disclosure.

FIG. 2 is a diagrammatic cross-sectional view of an ultrasound transducer according to an embodiment of the present disclosure.

FIGS. 3A-3F are diagrammatic cross-sectional views of the ultrasound transducer taken along line 2-2 in FIG. 2 according to various embodiments of the present disclosure.

FIG. 4 is a diagrammatic cross-sectional view of an ultrasound transducer according to another embodiment of the present disclosure.

FIG. 5 is a method for forming an ultrasound transducer, such as the ultrasound transducer of FIG. 2, according to various aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, the present disclosure provides an intravascular ultrasound (IVUS) imaging system described in terms of cardiovascular imaging, however, it is understood that such description is not intended to be limited to this application. The IVUS imaging system is equally well suited to any application requiring imaging within a small cavity. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

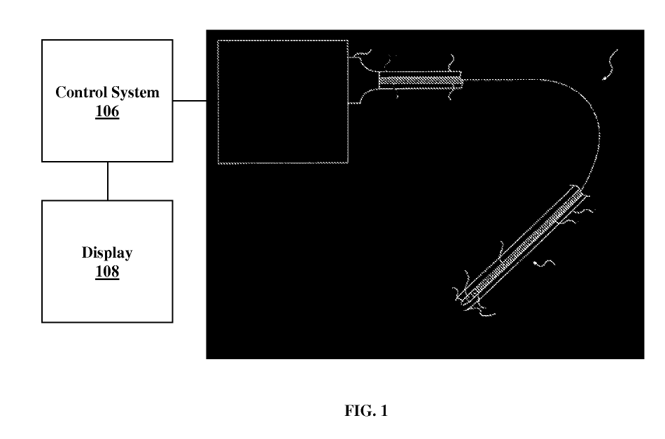

FIG. 1 is a schematic illustration of an intravascular ultrasound (IVUS) imaging system 100 according to various aspects of the present disclosure. The IVUS imaging system 100 includes an IVUS catheter 102 coupled by a patient interface module (PIM) 104 to an IVUS control system 106. The control system 106 is coupled to a monitor 108 that displays an IVUS image (such as an image generated by the IVUS system 100).

The IVUS catheter 102 is a rotational IVUS catheter, which may be similar to a Revolution® Rotational IVUS Imaging Catheter available from Volcano Corporation and/or rotational IVUS catheters disclosed in U.S. Pat. No. 5,243,988 and U.S. Pat. No. 5,546,948, both of which are incorporated herein by reference in their entirety. The catheter 102 includes an elongated, flexible catheter sheath 110 (having a proximal end portion 114 and a distal end portion 116) shaped and configured for insertion into a lumen of a blood vessel (not shown). A longitudinal axis LA of the catheter 102 extends between the proximal end portion 114 and the distal end portion 116. The catheter 102 is flexible such that it can adapt to the curvature of the blood vessel during use. In that regard, the curved configuration illustrated in FIG. 1 is for exemplary purposes and in no way limits the manner in which the catheter 102 may curve in other embodiments. Generally, the catheter 102 may be configured to take on any desired straight or arcuate profile when in use.

A rotating imaging core 112 extends within the sheath 110. The imaging core 112 has a proximal end portion 118 disposed within the proximal end portion 114 of the sheath 110 and a distal end portion 120 disposed within the distal end portion 116 of the sheath 110. The distal end portion 116 of the sheath 110 and the distal end portion 120 of the imaging core 112 are inserted into the vessel of interest during operation of the IVUS imaging system 100. The usable length of the catheter 102 (for example, the portion that can be inserted into a patient, specifically the vessel of interest) can be any suitable length and can be varied depending upon the application. The proximal end portion 114 of the sheath 110 and the proximal end portion 118 of the imaging core 112 are connected to the interface module 104. The proximal end portions 114, 118 are fitted with a catheter hub 124 that is removably connected to the interface module 104. The catheter hub 124 facilitates and supports a rotational interface that provides electrical and mechanical coupling between the catheter 102 and the interface module 104.

The distal end portion 120 of the imaging core 112 includes a transducer assembly 122. The transducer assembly 122 is configured to be rotated (either by use of a motor or other rotary device or manually by hand) to obtain images of the vessel. The transducer assembly 122 can be of any suitable type for visualizing a vessel and, in particular, a stenosis in a vessel. In the depicted embodiment, the transducer assembly 122 includes a piezoelectric micromachined ultrasonic transducer (“PMUT”) transducer and associated application-specific integrated circuit (ASIC). The transducer assembly 122 may include a housing having the PMUT transducer and associated circuitry disposed therein, where the housing has an opening that ultrasound signals generated by the PMUT transducer travel through.

The rotation of the imaging core 112 within the sheath 110 is controlled by the interface module 104, which provides user interface controls that can be manipulated by a user. The interface module 104 can receive, analyze, and/or display information received through the imaging core 112. It will be appreciated that any suitable functionality, controls, information processing and analysis, and display can be incorporated into the interface module 104. In an example, the interface module 104 receives data corresponding to ultrasound signals (echoes) detected by the imaging core 112 and forwards the received echo data to the control system 106. In an example, the interface module 104 performs preliminary processing of the echo data prior to transmitting the echo data to the control system 106. The interface module 104 may perform amplification, filtering, and/or aggregating of the echo data. The interface module 104 can also supply high- and low-voltage DC power to support operation of the catheter 102 including the circuitry within the transducer assembly 122.

In some embodiments, wires associated with the IVUS imaging system 100 extend from the control system 106 to the interface module 104 such that signals from the control system 106 can be communicated to the interface module 104 and/or visa versa. In some embodiments, the control system 106 communicates wirelessly with the interface module 104. Similarly, it is understood that, in some embodiments, wires associated with the IVUS imaging system 100 extend from the control system 106 to the monitor 108 such that signals from the control system 106 can be communicated to the monitor 108 and/or visa versa. In some embodiments, the control system 106 communicates wirelessly with the monitor 108.

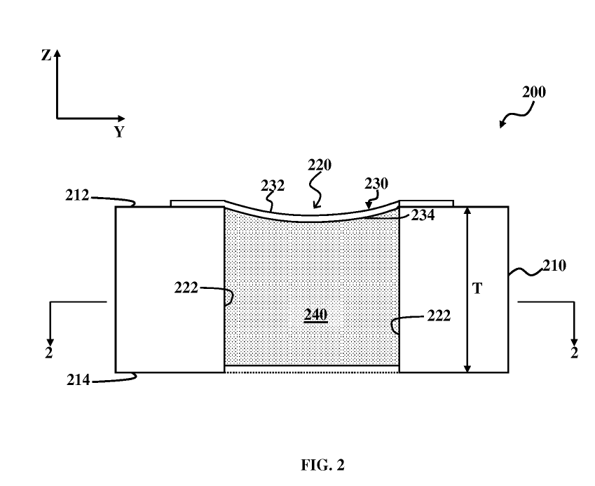

FIG. 2 is a diagrammatic cross-sectional view, particularly a Y-Z plane view, of an ultrasound transducer 200 according to an embodiment of the present disclosure. The ultrasound transducer 200 can be included in the IVUS imaging system 100 of FIG. 1, particularly in the transducer assembly 122. In the depicted embodiment, the ultrasound transducer 200 is a piezoelectric micromachined ultrasound transducer (PMUT). FIG. 2 has been simplified for the sake of clarity to better understand the inventive concepts of the present disclosure. Additional features can be added in the ultrasound transducer 200, and some of the features described below can be replaced or eliminated for additional embodiments of the ultrasound transducer 200.

The PMUT 200 includes a substrate 210 that has a surface 212 and a surface 214 that is opposite the surface 212. In the depicted embodiment, the substrate 210 is a silicon microelectromechanical system (MEMS) substrate. The substrate 210 includes another suitable material depending on design requirements of the PMUT transducer 200 in alternative embodiments. A well 220 is defined by a well surface 222 of the substrate 210, extending from the surface 212 to the surface 214. In the present example, the well 220 has a depth that is substantially equivalent to a thickness, T, of the substrate 210 (measured between the surface 212 and the surface 214).

A transducer membrane 230 is positioned over the well 220 adjacent to the first surface 212 of the substrate 210. The transducer membrane 230 may be partially or completely disposed within the well 220. The transducer membrane 230 has a surface 232 and a surface 234 that is opposite the surface 232 (here, the surface 232 may be referred to as a topside surface and the surface 234 may be referred to as a bottom side surface), where a thickness of the transducer membrane 230 is measured between the surface 232 and surface 234. In the depicted embodiment, the transducer membrane 230 has a shape configured to spherically focus ultrasound signals emitted therefrom. For example, the transducer membrane 230 has an arcuate shape that spherically focuses ultrasound signals emitted from the transducer membrane 230. The transducer membrane 230 may exhibit other shaped configurations to achieve various focusing characteristics. For example, in an alternative embodiment, the transducer membrane 230 has a substantially planar shape.

The transducer membrane 230 is a thin layer (film) of piezoelectric material. In some instances, the transducer membrane 230 has a thickness between 5 μm and 20 μm, with some particular emebodiments having a thickness between about 9 μm and about 15 μm. In the depicted embodiment, the transducer membrane 230 includes a piezoelectric polymer material, such as polyvinylidene fluoride (PVDF), polyvinylidene fluoride-trifluoroethylene (PVDF-TrFE), polyvinylidene fluoride-tetrafluoroethylene (PVDF-TFE), other piezoelectric polymer material, or combinations thereof.

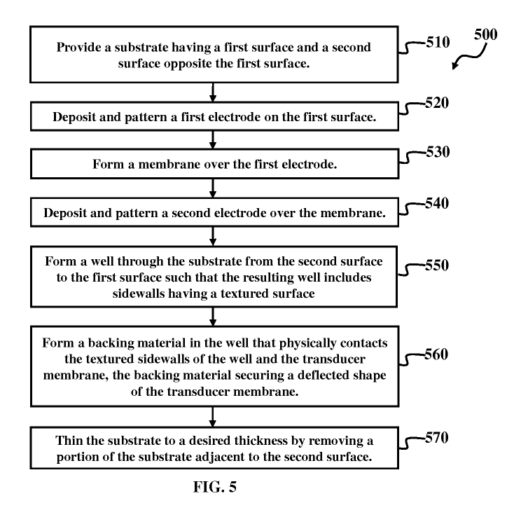

The transducer membrane 230 is electrically coupled to electronic circuitry. In an example, two electrodes (not shown) electrically couple the transducer membrane 230 to the electronic circuitry. In particular, in some instances an electrode is formed adjacent the surface 232 of the transducer membrane 230 and another electrode is formed adjacent the surface 234 of the transducer membrane 230. FIG. 4 illustrates an exemplary embodiment that includes an upper electrode 236 and a lower electrode 238. In some instances, the upper and lower electrodes associated with the transducer membrane 230 are electrically coupled to electronic circuitry as described in U.S. Provisional Patent Application Ser. No. 61/646,062, entitled “CIRCUIT ARCHITECTURES AND ELECTRICAL INTERFACES FOR ROTATIONAL INTRAVASCULAR ULTRASOUND (IVUS) DEVICES”, filed May 11, 2012, or U.S. Pat. No. 6,641,540, entitled “MINIATURE ULTRASOUND TRANSDUCER,” filed Sep. 6, 2001, each of which is hereby incorporated by reference in its entirety. The electronic circuitry 236 can excite the transducer membrane 230 so that it generates sound waves, particularly sound waves in an ultrasound range. The substrate 210 may includes various layers that are not separately depicted and that can combine to form the electrodes and/or other electronic circuitry that includes various microelectronic elements, which may include: transistors (for example, metal oxide semiconductor field effect transistors (MOSFET), complementary metal oxide semiconductor (CMOS) transistors, bipolar junction transistors (BJT), high voltage transistors, high frequency transistors, p-channel and/or n-channel field effect transistors (PFETs/NFETs)); resistors; diodes; capacitors; inductors; fuses; and/or other suitable elements. The various layers may include high-k dielectric layers, gate layers, hard mask layers, interfacial layers, capping layers, diffusion/barrier layers, dielectric layers, conductive layers, other suitable layers, or combinations thereof. The microelectronic elements could be interconnected to one another to form a portion of an integrated circuit, such as a logic device, memory device (for example, a static random access memory (SRAM)), radio frequency (RF) device, input/output (I/O) device, system-on-chip (SoC) device, other suitable types of devices, or combinations thereof.

A backing material 240 is disposed in the well 220. The backing material 240 physically contacts the well surface 222 of the substrate 210 and the surface 234 of the transducer membrane 230 (here, the backside surface of the transducer membrane 230). The backing material 240 locks the transducer membrane 230 into place such that its shape (here, the arcuate or spherical shape) is maintained. In that regard, in some instances the transducer membrane 230 is deflected between about 10 microns and 50 microns into the well 220, with some particular embodiments being deflected approximately 20 microns. The backing material 240 is an acoustically attenuative material so that the backing material 240 can absorb acoustic energy (in other words, sound waves) generated by the transducer membrane 230 that travel (propagate) into the ultrasound transducer 200 (for example, from the surface 234 of the transducer membrane 230 into the backing material 240), including acoustic energy that is reflected from structures and interfaces of a transducer assembly, such as when the ultrasound transducer 200 is included in the transducer assembly 122 of FIG. 1. In the present example, the backing material 240 is an epoxy composite material. The backing material 240 may include other materials that provide sufficient acoustical attenuation and mechanical strength for maintaining the shape of the transducer membrane 230. The backing material 240 may include a combination of materials for achieving such acoustical and mechanical properties. In some implementations the backing material is silver epoxy, tungsten-loaded epoxy, or epoxy loaded with other particulate materials, including high-density materials such as cerium oxide, tungsten oxide, aluminum oxide, and/or other suitable particulate materials.

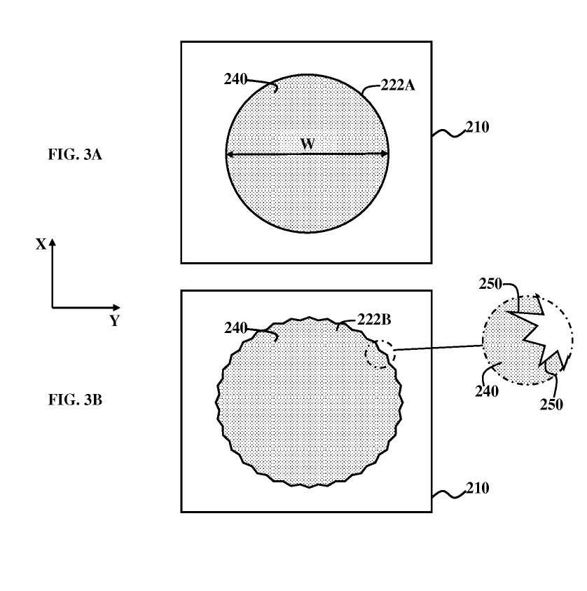

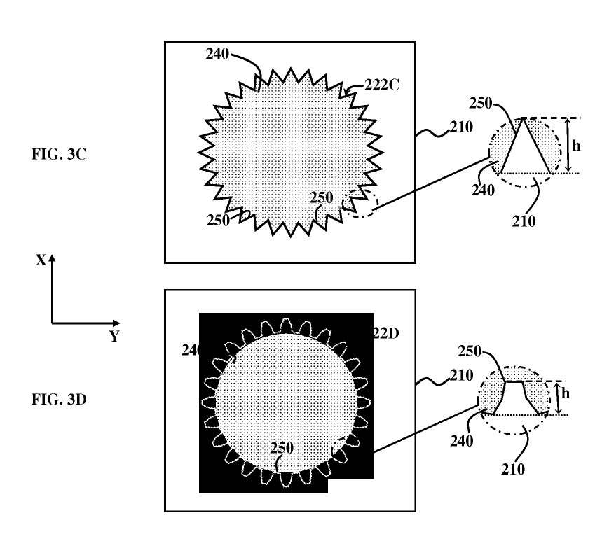

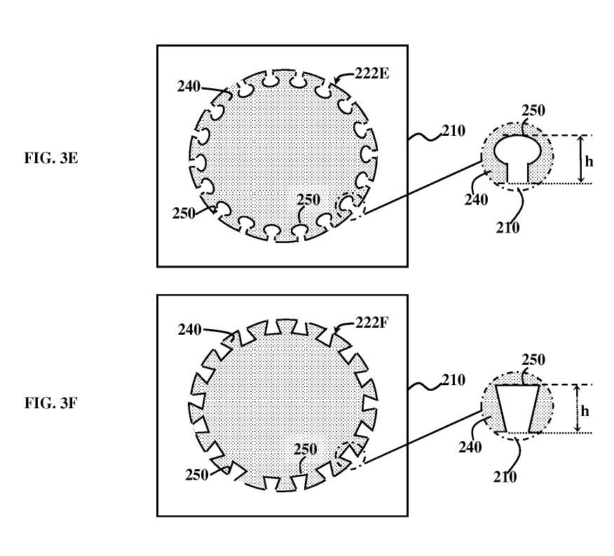

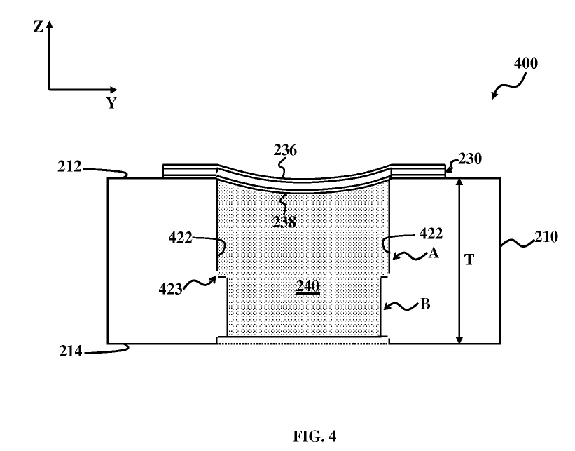

FIGS. 3A-3F are diagrammatic cross-sectional views, particularly in an X-Y plane, of the ultrasound transducer 200 taken along line 2-2 in FIG. 2 according to various embodiments of the present disclosure. In FIG. 3A, the well 220 is defined by a well surface 222A that is substantially smooth. In the depicted embodiment, the substantially smooth well surface 222A is cylindrically-shaped with a circular cross-sectional profile as shown in FIG. 3A. Alternatively, the substantially smooth well surface 222A may exhibit other shaped wells with other geometrically or non-geometrically shaped cross-sectional profiles. It has been observed that when the well 220 is defined by substantially smooth surfaces of the substrate 210, such as the well surface 222A, the backing material 240 has a tendency to become dislodged or otherwise move relative to the substrate 210. Such movement of the backing material 240 presents issues during manufacturing and operation of the ultrasound transducer 200. For example, during manufacturing of the ultrasound transducer 200, portions of the backing material 240 intended to stay within the well 220 can be undesirably removed from the well 220 because of movement of the backing material 240 relative to the substrate 210. In a particular example, portions of the backing material 240 are removed from the well 220 during a grinding process that thins the substrate 210, sometimes being displaced by the slurry used during the grinding process. Further, during operation of the ultrasound transducer 200, portions of the backing material 240 may dislodge from the substrate 210, sometimes falling out of the well 220. These issues result in the complete failure of the ultrasound transducer 200, and therefore, the inability to use the transducer in any device or system.

The present disclosure thus proposes modifying the well surface 222A illustrated in FIG. 3A, such that the well 220 is defined by a textured surface of the substrate 210, such as the textured well surfaces 222B, 222C, 222D, 222E, and 222F respectively illustrated in FIGS. 3B-3F. When compared to the well surface 222A (a cylindrically-shaped well surface having a circular cross-sectional profile), a textured surface of the substrate 210 provides increased bonding surface area between the backing material 240 and the substrate 210 and facilitates mechanical interlocking between the backing material 240 and the substrate 210. The greater bonding surface area and mechanical interlocking provided by the textured surfaces of the present disclosure improve securing of the backing material 240 within the well 220. For example, the various textured well surfaces 222B, 222C, 222D, 222E, and 222F include projections 250 that extend from the substrate 210 into the well 220 to facilitate increased bonding area and a mechanical locking interface between the substrate 210 and the backing material 240. A configuration of the projections 250 is optimized to (1) maximize surface area of the substrate 210 for bonding with the backing material 240; (2) provide a mechanical interlock between the backing material 240 and the substrate 210; (3) provide an easy pathway for impregnating the backing material 240, such as epoxy, that prevents or reduces bubble trapping or voids within the backing material 240; (4) provide adequate support for the transducer membrane 230 (particularly at edges of the transducer membrane 230 that physically contact the substrate 210), such that a desired shape (here, the arcuate shape) of the transducer membrane 230 is maintained; (5) minimize manufacturing costs and time, (6) other considerations; or (7) a combination thereof.

The projections 250 of the various textured well surfaces 222B, 222C, 222D, 222E, and 222F exhibit cross-sectional profiles having a two-dimensional pattern in the X-Y plane views. The projections 250 may exhibit a non-periodic two-dimensional pattern, such as that illustrated in FIG. 3B, or a periodic two-dimensional pattern (where a spacing between the respective centers of adjacent projections is equal), such as that illustrated in FIGS. 3C and 3D. The projections 250 exhibit geometrical shapes, non-geometrical shapes, or combinations thereof. For example, the projections 250 exhibit a triangular shape (FIG. 3C), a trapezoidal shape (FIG. 3F), a rectangular shape, an arcuate shape, a cross shape, other shape (such as that illustrated in FIG. 3D), or combination of shapes (such as that illustrated in FIG. 3E, where the projections 250 include a spherical-shaped portion and a rectangular-shaped portion). The illustrated projections 250 are not intended to be limiting, and it is understood that any appropriately shaped projection is contemplated by the present disclosure. Further, the array of projections 250 can alternatively include projections with various shapes.

The well surfaces 222B, 222C, 222D, 222E, and 222F include texture provided by the projections 250, the textured well surfaces 222B, 222C, 222D, 222E, and 222F result in substantially cylindrically-shaped well surfaces having substantially circular cross-sectional profiles, yet the textured well surfaces 222B, 222C, 222D, 222E, and 222F provide increased surface area compared to the smooth well surface 222A that is cylindrically-shaped with a circular cross-sectional profile. Similarly, a smooth well surface that is rectangular-shaped with a rectangular cross-sectional profile provides less bonding surface area than a textured well surface that is substantially rectangular-shaped with a substantially rectangular cross-sectional profile, and so forth for other shaped well surfaces. In the depicted embodiments, a height (h) of each projection 250 is measured between a peak and a valley of the projection 250. In some embodiments, for the textured well surfaces 222B, 222C, 222D, 222E, and 222F to exhibit the substantially cylindrically-shaped well surfaces having substantially circular cross-sectional profiles, a mean peak-to-valley height (R_{z}) of the projections 250 is less than about 10 μm. In some embodiments, a surface roughness average (Rₐ) (which indicates a mean value of a height of the projections 250 relative to a center line average) or a root mean square roughness (R_{q}), is less than about 10 μm. In the present example, the center line average is defined by the circular profile of the smooth well surface 222A, such that the well 220 has a width (or diameter). In an example, the width is about 500 μm. In some examples, a ratio of the height of the projections 250 to the width of the well 220 is less than 1/50. In some implementations, the projections 250 have a peak to trough size between about 5 μm and about 15 μm. In that regard, in some instances the size of the projections 250 is selected based on the particle size in the backing material 240. For example, the size of the projections 250 may be selected such that 1-5 particles may be received between each projection. To that end, the particles of the backing material 240 have a size between about 2 μm and about 5 μm in some implementations.

FIG. 4 is a diagrammatic cross-sectional view of an ultrasound transducer 400 according to another embodiment of the present disclosure. The embodiment of FIG. 4 is similar in many respects similar to the embodiment of FIG. 2. For example, the ultrasound transducer 400 is a PMUT transducer, similar to the ultrasound transducer 200 of FIG. 2. Accordingly, similar features in FIGS. 2 and 4 are identified by the same reference numerals for clarity and simplicity. The ultrasound transducer 400 includes the substrate 210 having the surface 212 and the surface 214 opposite the surface 212. In the depicted embodiment, the substrate 210 includes a well 420 having a profile different than the well 220 illustrated in FIG. 2. For example, the well 420 is defined by a well surface 422 of the substrate 210. The well 420 extends from the surface 212 to the surface 214 and has a depth that is substantially equivalent to the thickness (T) of the substrate 210 (measured between the surface 212 and the surface 214). In contrast to the well surface 222, the well surface 422 has a portion A that tapers into a portion B via shelf 423. The portion A has a width greater than the portion B. Similar to the well 220 of the ultrasound transducer 200, the well surface 422 is a textured surface, such as those illustrated in FIGS. 3B-3F, where portion A of the well surface 422 is textured, portion B of the well surface 422 is textured, or both portion A and portion B are textured. Alternatively, the well surface 422 may be a non-textured surface, such as that illustrated in FIG. 3A, or include portions that are non-textured (such as portion A or portion B). Further, similar to the ultrasound transducer 200, the transducing membrane 230 is disposed in the well 420 adjacent to the surface 212 of the substrate 210, and the backing material 240 is also disposed within the well 420. The shelf 423 of the well 420 further secures the backing material 240 within the well 420. Additional features can be added in the ultrasound transducer 400, and some of the features described can be replaced or eliminated for other embodiments of the ultrasound transducer 400.

As shown in FIG. 4, the electrodes 236 and 238 include a bend or transition to accommodate the deflection of the membrane 230 into the well 220. However, in some instances where a purely cylindrical well with smooth walls is utilized, the stress of the associated bend or transition causes one or both of the electrodes 236 and 238 to crack or otherwise become damaged at the point where the electrode crosses the boundary into the well, resulting in a failure of the transducer. To this end, the textured surfaces of the wells of the present disclosure allow the stress associated with the bend or transition to be distributed over a larger area of the electrodes 236 and 238 as a result of the irregular well shape outline. Accordingly, in addition to improving the engagement with the backing material, the textured wells of the present disclosure also increase manufacturing efficiencies by reducing and/or eliminating electrode cracking due to transitions at the boundary of the well.

FIG. 5 is a flow chart of a method 500 for forming an ultrasound transducer, such as the ultrasound transducer 200 of FIG. 2 or the ultrasound transducer 400 of FIG. 4, according to various aspects of the present disclosure. The ultrasound transducer fabricated by the method 500 improves bonding of a backing material to a surface of a substrate that defines a well therein. The method 500 begins at block 510 where a substrate having a first surface and a second surface opposite the second surface is provided. The substrate may be similar to the substrate 210 described above, for example, the substrate is a silicon, MEMS substrate. At block 520, a first electrode, such as electrode 238 shown in FIG. 4, is deposited, patterned, and/or otherwise formed on the first surface of the substrate. At block 530, a transducing membrane is formed over the first electrode adjacent the first surface of the substrate. The transducing membrane may be similar to the transducing membrane 230 described above. The transducing membrane is formed by any suitable process, for example, a piezoelectric material layer is deposited over the first surface of the substrate and patterned to form the transducing membrane. At this point, the transducing membrane is substantially planar. At block 540, a second electrode, such as electrode 236 shown in FIG. 4, is deposited, patterned, and/or otherwise formed on the transducing membrane.

At block 550, a portion of the substrate is removed, thereby exposing the first electrode and/or the transducing membrane. For example, a backside portion of the substrate 210 is removed, such that the electrode 238 and/or the surface 234 of the transducing membrane 230 is exposed from surface 214 of the substrate 210. A shape of the well is defined by a well surface of the substrate, and the well surface has a greater surface area than a cylindrically-shaped well surface. More specifically, the well surface is shaped and/or textured, such as that described above, with reference to FIGS. 2, 3B-3F, and 4. The portions of the substrate are removed by a micromachining process. In some instances, the texture of the well surface is defined by a photolithography process. In the present example, the micromachining process includes a dry etching process, specifically a deep reactive ion etching (DRIE) process. In that regard, DRIE provides precise etching in a vertical direction according to the photolithography pattern. In some instances, the DRIE process comprises a plurality of alternating steps of etching away a portion of the substrate, and depositing a polymer to protect the sidewalls already formed, and etching away additional portions of the substrate (including polymer deposited on surface extending across the well). The etching process has etching parameters that are tuned, such as etchants used, etching pressure, source power, radio-frequency (RF) bias voltage, RF bias power, etchant flow rate, and other suitable parameters, to achieve the textured well surface. In an example, the dry etching process uses a patterned resist layer as a mask to achieve the desired texturing of the well surface. The patterned resist layer is formed by a lithography process that may include photoresist coating (e.g., spin-on coating), soft baking, mask aligning, exposure, post-exposure baking, developing the photoresist, rinsing, drying (e.g., hard baking), other suitable processes, or combinations thereof. Alternatively, the lithography exposing process may include maskless photolithography, electron-beam writing, or ion-beam writing. In yet another alternative, the desired well texturing is achieved by performing a wet etching process, or a combination of dry and wet etching processes. For example, a multi-step etching process forms a well having a shelf, such as the well 422 having the shelf 423 illustrated in FIG. 4. The multi-step etching process may use a combination of anisotropic and isotropic wet etching processes to achieve the desired well profile, such as the shelf 423. Further, while DRIE process is typically performed to intentionally form a precisely vertical pattern, in some instances the parameters are adjusted to ensure that you don't get a vertical wall, but instead get a surface that is tapered and/or textured in a vertical direction in addition to or in lieu of the texture extending circumferentially around the well.

After the well is formed, a process is performed to deflect the substantially planar shape of the transducing membrane and associated electrodes. For example, the transducing membrane is deflected such that the transducing membrane has an arcuate, bowl shape. The arcuate shape of the transducing membrane can be achieved by methods described in U.S. Pat. No. 6,641,540, hereby incorporated by reference in its entirety. At block 560, a backing material is formed in the well that physically contacts the well surface and the transducing membrane, such that the backing material secures the transducer membrane in the desired shape, such as the arcuate shape. The etching process or processes used to form the well may leave a polymer residue (such as a fluoropolymer residue) along the well surface, which in some instances, is removed before forming the backing material. As discussed above, the textured well surface provides mechanical interlocking between the backing material and the substrate, securely confining the backing material within the well, and improving the backing material's securing function of the arcuate shape of the transducing membrane. In that regard, at block 570 the substrate is thinned to a desired thickness by removing a portion of the substrate adjacent to the second surface. The desired thickness is in the range of 50 μm to 200 μm in some implementations, with some particular embodiments having a thickness of approximately 75 μm. To that end, in some embodiments the substrate, before thinning, has a thickness between about 200 μm and about 1,000 μm, with some particular embodiments having a thickness of approximately 400 μm. In some instances, the portions of the backside of the substrate (such as the surface 214 of the substrate 210) are removed using a grinding process, polishing process, other substrate removal process, or combination thereof). The textured well surface helps to ensure that the backing material remains intact within the well during the thinning process.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An ultrasound transducer comprising:
a substrate having a first surface and a second surface opposite the first surface;
a well disposed in the substrate that extends from the first surface to the second surface, wherein a well surface of the substrate defines a shape of the well, and further wherein the well surface has a greater surface area than a cylindrically-shaped well surface;
an arcuate-shaped transducer membrane positioned within the well adjacent to the first surface of the substrate; and
a backing material disposed in the well that physically contacts the well surface and the transducer membrane such that the backing material secures the arcuate-shape of the transducer membrane.

2. The ultrasound transducer of claim 1 wherein the well surface is a textured surface of the substrate, wherein the textured surface includes projections that provide mechanical interlocking between the backing material and the substrate.

3. The ultrasound transducer of claim 2 wherein the projections have heights less than about 10 μm.

4. The ultrasound transducer of claim 2 wherein the projections in a two-dimensional plane have a periodic structure.

5. The ultrasound transducer of claim 2 wherein the projections in a two-dimensional plane have a non-periodic structure.

6. The ultrasound transducer of claim 2 wherein the projections have a geometrical shape or combination of geometrical shapes.

7. The ultrasound transducer of claim 1 wherein the arcuate-shaped membrane is configured to spherically focus ultrasound signals emitted therefrom.

8. The ultrasound transducer of claim 1 wherein the substrate is a silicon substrate.

9. The ultrasound transducer of claim 1 wherein the substrate is a microelectromechanical system (MEMS) substrate.

10. The ultrasound transducer of claim 1 wherein the membrane includes a piezoelectric material.

11. The ultrasound transducer of claim 10 wherein the piezoelectric material is polyvinylidenefluroide trifluoroethylene (pVDF-TrFE).

12. The ultrasound transducer of claim 1 wherein the backing material is an epoxy.

13. A method for fabricating an ultrasound transducer, the method comprising:
providing a substrate having a first surface and a second surface opposite the first surface;
forming a well in the substrate that extends from the first surface to the second surface, wherein a shape of the well is defined by a well surface of the substrate, and further wherein the well surface has a greater surface area than a cylindrically-shaped well surface;
forming a transducing membrane adjacent to the first surface of the substrate, wherein the transducing membrane is disposed within the well; and
forming a backing material in the well that physically contacts the well surface and the transducer membrane such that the backing material secures a shape of the transducer membrane.

14. The method of claim 13 further including thinning the substrate after forming the backing material.

15. The method of claim 14 wherein the thinning the substrate includes performing a grinding process on the second surface of the substrate.

16. The method of claim 13 wherein the forming the well in the substrate includes performing a deep reactive ion etching (DRIE) process.

17. The method of claim 16 wherein forming the well in the substrate includes forming a patterned resist layer over the second surface of the substrate, the patterned resist layer defining a pattern of the well surface, wherein the DRIE process uses the patterned resist layer as a mask.

18. The method of claim 13 further including removing a polymer residue from the well surface before forming the backing material.

19. The method of claim 13 wherein the forming the transducing membrane includes modifying a planar shape of the transducing membrane, such that the transducing membrane has an arcuate shape, after forming the well in the substrate, wherein the backing material secures the arcuate shape of the transducer membrane.

20. The method of claim 19 wherein the forming the well in the substrate includes forming a textured surface that includes projections that extend into the well, wherein the projections provide mechanical interlocking between the backing material and the substrate.

21. An intravascular ultrasound (IVUS) system, comprising:
an elongate flexible member having a proximal end portion and a distal end portion;
a piezoelectric micromachined ultrasound transducer (PMUT) coupled to the distal end portion of the flexible elongate member, wherein the PMUT includes:
a substrate having a first surface and a second surface opposite the first surface,
a well disposed in the substrate that extends from the first surface to the second surface, wherein a well surface of the substrate defines a shape of the well, and further wherein the well surface has a greater surface area than a cylindrically-shaped well surface,
an arcuate-shaped transducing membrane positioned within the well adjacent to the first surface, and
a backing material disposed in the well that physically contacts the well surface and the membrane such that the backing material secures the arcuate-shape of the transducing membrane; and
an integrated circuit coupled to the distal end portion of the flexible elongate member and electrically coupled to the PMUT.

22. The system of claim 21, further comprising: an interface module configured to engage with the proximal end portion of the flexible elongate member.

23. The system of claim 22, further comprising: an image processing component in communication with the interface module.
